(19) United States
(12) Patent Application Publication
(10) Pub. No.: US 2026/0325356 A1
(43) Pub. Date: Oct. 22, 2026

(54) EAR TIP OF ACOUSTIC DEVICE

(71) Applicant: SHENZHEN QIYUAN ZHIHUI TECHNOLOGY CO., LTD., SHENZHEN CITY (CN)

(72) Inventor: JIA CHEN, SHENZHEN CITY (CN)

(21) Appl. No.: 19/406,957

(22) Filed: Dec. 3, 2025

(30) Foreign Application Priority Data
Apr. 18, 2025 (CN) 202510494563.9

(51) Int. Cl. A61F 11/10

(52) U.S. Cl. CPC A61F 11/10; A61F 2230/0067; A61F 2230/008

(57) ABSTRACT

The present invention relates to an ear tip of an acoustic device, and belongs to the technical field of earplugs. The ear tip includes a cap body and a support member. The cap body is made of a flexible material, a first cavity is formed inside the cap body, a through hole connected to the first cavity is provided at one end of the cap body close to an ear canal of a user, an arc-shaped transition portion is formed between one end of the cap body close to the ear canal of the user and a side end connected to the cap body, and one end of the cap body distal from the ear canal of the user is provided with a supporting protrusion; and the support member contacts the supporting protrusion and a second cavity is formed between the support member and the supporting protrusion.

### CROSS-REFERENCE TO RELATED PATENT APPLICATION

The present disclosure claims priority to Chinese Patent Application No. 2025104945639, entitled "EAR TIP OF ACOUSTIC DEVICE", filed with the China National Intellectual Property Administration on Apr. 18, 2025, the entire contents of which are incorporated herein by reference.

### FIELD OF THE DISCLOSURE

The present invention relates to the technical field of earplugs, and more particularly, to an ear tip of an acoustic device.

### BACKGROUND OF THE DISCLOSURE

In daily life, work, or commuting, people are often exposed to environments with high-intensity or continuous noise, such as traffic, mechanical equipment, or noisy crowds. Long-term exposure to such noise environments may cause hearing fatigue or even hearing damage. Earplugs physically isolate external noise and provide a quiet environment for users, thereby reducing noise hazards at the source. For individuals who are easily affected by noise, such as light sleepers or people living in noisy surroundings, earplugs can effectively block environmental noise, assist in falling asleep, prolong the duration of deep sleep, and improve overall sleep quality.

To reduce the impact of noise on daily life, noise-reducing earplugs have become the most common choice. However, most noise reduction earplugs on the market are in a standard circular shape, which provides poor adaptability to differences in ear canal structures of users, resulting in uncomfortable wearing experiences.

### SUMMARY OF THE DISCLOSURE

The technical problem to be solved by the present invention is that most noise reduction earplugs on the market are in a standard circular shape, which provides poor adaptability to differences in ear canal structures of users and results in uncomfortable wearing experiences. In view of the above defects in the prior art, the present invention provides an ear tip of an acoustic device.

To solve the above technical problem, the present invention adopts the following technical solutions.

An ear tip of an acoustic device includes:
a cap body, where the cap body is made of a flexible material, a first cavity is formed inside the cap body, a through hole connected to the first cavity is provided at one end of the cap body close to an ear canal of a user, and an arc-shaped transition portion is formed between one end of the cap body close to the ear canal of the user and a side end connected to the cap body.

Optionally, a supporting protrusion is provided at one end of the cap body distal from the ear canal of the user;
the ear tip includes a support member, where the support member is in contact with the supporting protrusion, and a second cavity is formed between the support member and the supporting protrusion, so that air within the second cavity is expelled in a case where the supporting protrusion is pressed by an external force;
and one end of the supporting protrusion distal from the cap body is provided with a leak-preventing contact portion configured to increase a contact area with the support member.

Optionally, a first included angle between the leak-preventing contact portion and the supporting protrusion is 110 degrees to 160 degrees to form an outwardly expanding structure.

Optionally, the ear tip includes at least a flexible supporting outer layer and a sound-insulating inner layer which are stacked, and the supporting protrusion is made of the same material as the flexible supporting outer layer and is integrally formed with the flexible supporting outer layer.

Optionally, a sealing buffer layer is arranged between the supporting protrusion and the flexible supporting outer layer, and the sealing buffer layer is configured to fill gaps, enhance structural integrity, and achieve effective air tightness by fitting a contact interface, so that sound wave leakage is prevented.

Optionally, one end of the cap body distal from the ear canal of the user is provided with a supporting protrusion, and an inner wall of the supporting protrusion is provided with a plurality of elastic supporting portions in contact with the support member to provide a supporting function.

Optionally, a clamping base is arranged at one end of the through hole close to the first cavity, and a dustproof member or a noise reduction member is clamped onto the clamping base.

Optionally, the clamping base includes a hollow cylindrical mounting portion protruding from an interior of the first cavity, an outer peripheral side of the hollow cylindrical mounting portion is provided with a clamping protrusion, and a peripheral side of the dustproof member or the noise reduction member is provided with a clamping groove adapted to the clamping protrusion.

A noise reduction earplug includes the ear tip of an acoustic device, a noise reduction device, and a wearing portion, where the wearing portion is arranged at one end of the noise reduction device distal from the cap body, and one end of the first cavity distal from the through hole is provided with an assembly hole configured for connection with the noise reduction device.

Optionally, the support member has a hollow frustoconical shape and is integrally formed with the cap body or the noise reduction device.

The beneficial effects of the present invention are as follows:

The cap body is made of a flexible material, and a first cavity serving a supporting function is arranged inside the cap body. In a case where the ear tip is inserted into an ear canal, an appropriate deformation occurs due to the flexible structure, thereby providing a supporting function and preventing the cap body from collapsing, while buffering and stabilizing ear pressure to improve wearing comfort. An arc-shaped transition portion is formed between one end of the cap body close to the ear canal and a side end. This structure forms a smooth curve at a shape transition position to reduce local compression of an ear canal wall during insertion of the ear tip into the ear canal and to improve fitting and comfort during wearing.

### BRIEF DESCRIPTION OF THE DRAWINGS

To more clearly describe the technical solutions of the embodiments of the present invention or the prior art, the present invention is further illustrated below in conjunction with the accompanying drawings and embodiments. The accompanying drawings described below are merely some embodiments of the present invention. Those skilled in the art may obtain other accompanying drawings based on these accompanying drawings without any inventive effort.

FIG. 1 is an overall isometric schematic diagram of an embodiment of the present invention.

FIG. 2 is a cross-sectional schematic diagram of an embodiment of the present invention taken along line A-A in FIG. 1.

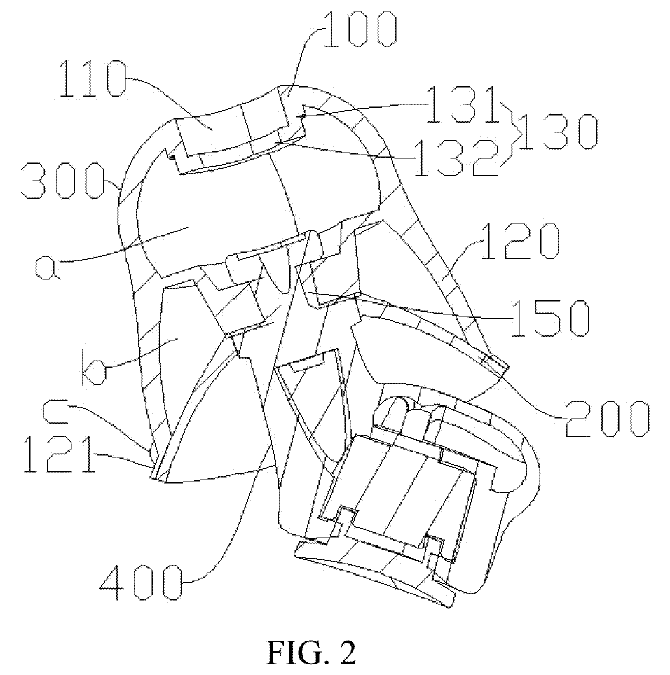

FIG. 3 is a front schematic diagram of an embodiment of the present invention.

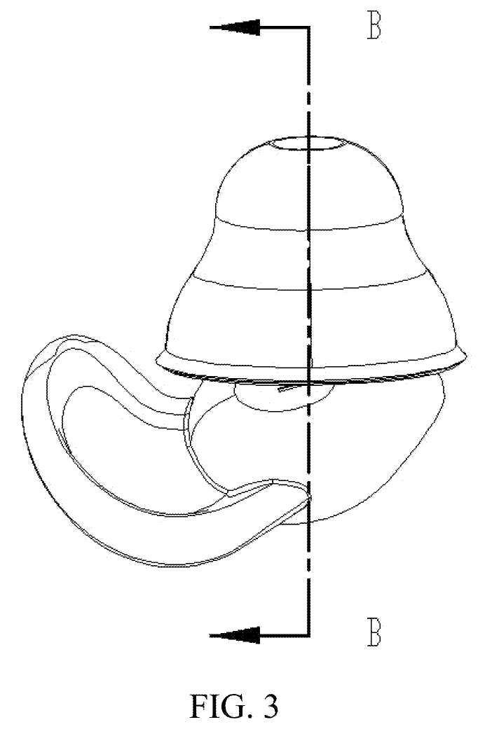

FIG. 4 is a cross-sectional schematic diagram of an embodiment of the present invention taken along line B-B in FIG. 3.

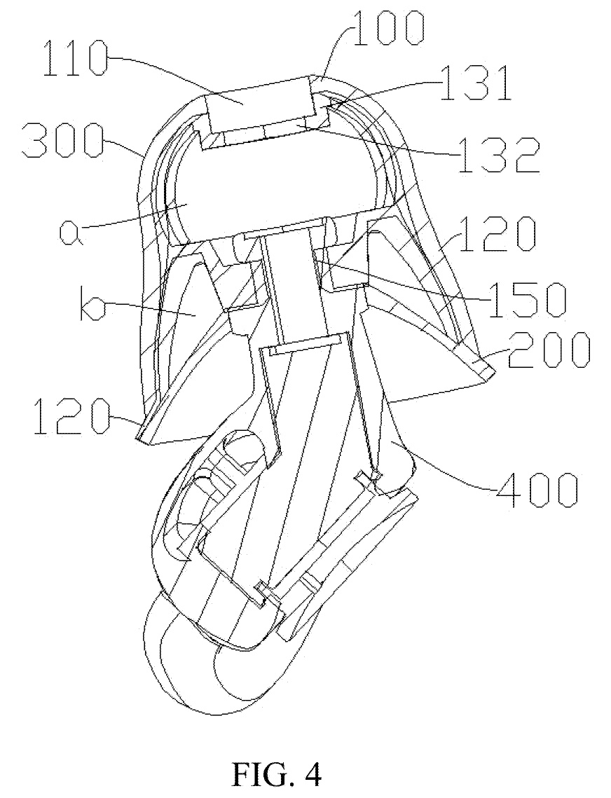

FIG. 5 is an overall exploded schematic diagram of an embodiment of the present invention.

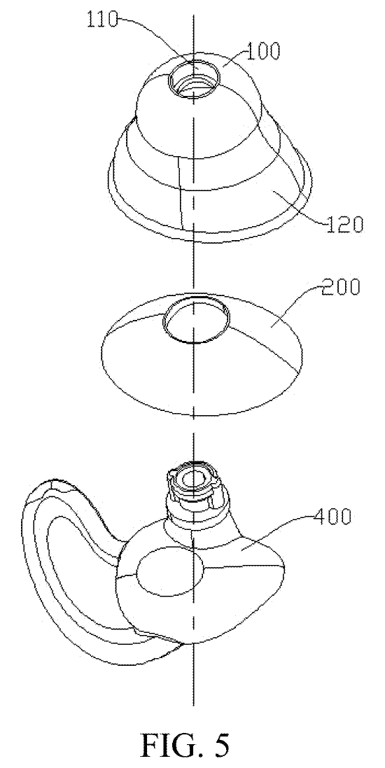

FIG. 6 is a cross-sectional schematic diagram of Embodiment 3 of the present invention.

### REFERENCE NUMERALS

100. cap body; 110. through hole; 120. supporting protrusion; 121. leak-preventing contact portion; 130. clamping base; 131. hollow cylindrical mounting portion; 132. clamping protrusion; 140. noise reduction member; 141. clamping groove; 150. assembly hole; 160. inclined supporting portion; 200. support member; 300. arc-shaped transition portion; 400. noise reduction device; 500. wearing portion; a. first cavity; b. second cavity; c. first included angle; d. second included angle; and e. distance between a top and a bottom of a first cavity.

### DETAILED DESCRIPTION OF THE EXEMPLARY EMBODIMENTS

To make objectives, technical solutions, and beneficial effects of the present invention clearer, the following clearly and completely describes the technical solutions of the present invention with reference to the accompanying drawings of the present invention. It is clear that the described embodiments are some but not all of embodiments of the present invention. All other embodiments obtained by those of ordinary skill in the art based on embodiments of the present invention without creative efforts shall fall within the protection scope of the present invention.

It should be noted in the description of the embodiments of the present application that, unless otherwise clearly specified and defined, the terms "interconnect" and "connect" should be understood in a broad sense. For example, such terms may indicate a fixed connection, a detachable connection, or an integral connection; may indicate a mechanical connection or an electrical connection; and may indicate direct interconnection, indirect interconnection through an intermediate medium. Those of ordinary skill in the art may understand specific meanings of the foregoing terms in the embodiments of the present invention based on a specific situation.

In the embodiments of the present invention, unless otherwise specified and limited, when a first feature is "above" or "below" a second feature, the first feature may be in direct contact with the second feature, or the first feature may be in indirect contact with the second feature through an intermediate medium. In addition, that the first feature is "on", "above" or "over" the second feature may be that the first feature is right above or obliquely above the second feature, or merely means that a horizontal height of the first feature is greater than that of the second feature. That the first feature is "below", "under" or "beneath" the second feature may be that the first feature is right below or obliquely below the second feature, or merely means that a horizontal height of the first feature is less than that of the second feature.

In the description of this specification, the description with reference to the terms "one embodiment", "some embodiments", "example", "specific example", "some examples" or the like means the particular features, structures, materials or characteristics described in combination with the embodiment or example are included in at least one embodiment or example of the present invention. In this specification, the schematic expression of the above terms does not necessarily refer to the same embodiments or examples. Furthermore, the specific features, structures, materials, or characteristics described can be combined in any suitable manner in any one or more embodiments or examples. Moreover, various embodiments or examples and features of various embodiments or examples described in this specification may be combined and combined by one skilled in the art without mutual contradiction.

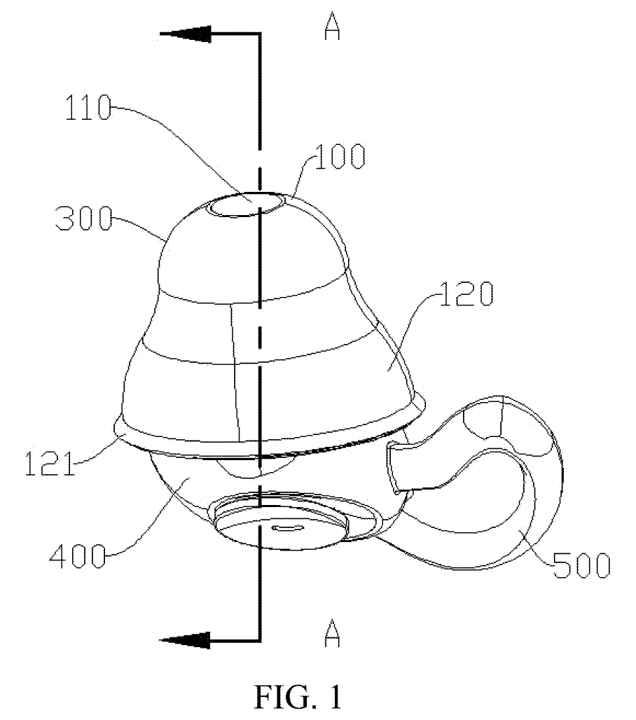

Embodiment 1: An embodiment of the present invention is shown in FIGS. 1 - 5 and relates to an ear tip of an acoustic device. The ear tip includes a cap body 100 and a support member 200. The cap body 100 is made of a flexible material, a first cavity a is formed inside the cap body, a through hole 110 connected to the first cavity is provided at one end of the cap body 100 close to an ear canal of a user, an arc-shaped transition portion is formed between one end of the cap body 100 close to the ear canal of the user and a side end connected to the cap body, and one end of the cap body 100 distal from the ear canal of the user is provided with a supporting protrusion 120. The support member 200 is in contact with the supporting protrusion 120, and a second cavity b is formed between the support member 200 and the supporting protrusion 120, so that air within the second cavity b is expelled in a case where the supporting protrusion 120 is pressed by an external force. Further, the cap body 100 is made of a flexible material, and the first cavity a serving a supporting function is arranged inside the cap body. In a case where the ear tip is inserted into the ear canal, an appropriate deformation occurs due to the flexible structure, thereby providing a supporting function and preventing the cap body 100 from collapsing, while buffering and stabilizing ear pressure to improve wearing comfort. One end of the cap body 100 close to the ear canal of the user is provided with a through hole 110 communicating with the first cavity a, so as to ensure entry of sound and avoid stimulation of the eardrum of the user caused by a fully enclosed structure. An arc-shaped transition portion 300 is formed between one end of the cap body 100 close to the ear canal and a side end of the cap body. This structure forms a smooth curve at a shape transition position to reduce local compression of an ear canal wall during insertion of the ear tip into the ear canal and to improve fitting and comfort during wearing. One end of the cap body 100 distal from the ear canal of the user is provided with a supporting protrusion 120 protruding outward. The supporting protrusion 120 is a connecting structure between the cap body 100 and the support member 200, which has a certain structural rigidity and elastic recovery capability, is capable of deforming under an external force, and forms a stable fitting structure with the support member 200, so as to provide deformation support and buffering function during wearing, thereby enhancing wearing stability of the ear tip structure.

To enhance stability of the ear tip, one end of the supporting protrusion 120 distal from the cap body 100 is provided with a leak-preventing contact portion 121 for increasing a contact area with the support member 200. Further, the leak-preventing contact portion 121 is capable of forming a larger fitting area with a surface of the support member 200, thereby improving the sealing effect between structures, preventing air leakage in a case where the second cavity b is not compressed, and enhancing overall airtightness and noise reduction performance of the ear tip. Further, the leak-preventing contact portion 121 may have an annular expansion structure or an inclined structure, and form a continuous fitting region with the support member 200 in a fitting state. In a case where the ear tip is pressed by an external force during wearing, the second cavity b changes volume due to structural deformation, and air in the second cavity b is expelled from the leak-preventing contact portion, thereby generating a buffering effect, reducing impact of instantaneous pressure change in the ear canal on the eardrum, and improving comfort and safety during wearing.

In this embodiment, a first included angle c between the leak-preventing contact portion 121 and the supporting protrusion 120 is 110 degrees to 160 degrees to form an outwardly expanding structure. Further, in this embodiment, a first included angle c between the leak-preventing contact portion 121 and the supporting protrusion 120 is 120 degrees. In some embodiments, the first included angle c between the leak-preventing contact portion 121 and the supporting protrusion 120 may also be 130 degrees, 140 degrees, or the like.

Referring to FIGS. 1 -5, in this embodiment, the ear tip includes at least a flexible supporting outer layer and a sound-insulating inner layer which are stacked, and the supporting protrusion 120 is made of the same material as the flexible supporting outer layer and is integrally formed with the flexible supporting outer layer. The flexible supporting outer layer is configured to provide overall structural strength and elastic supporting performance of the ear tip, and the sound-insulating inner layer is made of a material having excellent acoustic wave blocking performance and is configured to enhance the passive noise reduction effect of the ear tip. Preferably, the supporting protrusion 120 and the flexible supporting outer layer are made of the same material and integrally formed by an integral molding process, so as to ensure consistency between the supporting protrusion and the flexible supporting outer layer in mechanical properties and deformation response, enhance overall strength and stability of the ear tip, and avoid structural delamination or poor fitting caused by material differences. The flexible supporting outer layer may be made of silicone, thermoplastic polyurethane, or the like, and the sound-insulating inner layer may be made of foamed polyurethane, rubber, or the like.

In this embodiment, a sealing buffer layer is arranged between the supporting protrusion 120 and the flexible supporting outer layer, the sealing buffer layer is configured to fill gaps, enhance structural integrity, and achieve effective sealing by fitting a contact interface, thereby enhancing overall structural integrity and stability of the ear tip.

In this embodiment, the cap body 100 has a frustoconical shape or a nipple-like shape. Further, designing the cap body 100 with a frustoconical shape or a nipple-like shape facilitates smooth insertion of the ear tip into the ear canal of the user and enables flexible fitting with the ear canal wall during wearing through a gradually varying structure. The nipple-like structure further forms a smooth transition head on the basis of the frustoconical shape, and thus has stronger deformation adaptability.

Referring to FIGS. 1 -5, in this embodiment, a clamping base 130 is arranged at one end of the through hole 110 close to the first cavity, and a dustproof member or a noise reduction member 140 is clamped onto the clamping base 130. Further, the dustproof member is configured to block foreign matter, dust, or impurities from entering the first cavity a, so as to protect cleanliness and durability of internal structures; and the noise reduction member 140 may be a physical sound-absorbing component or a filtering material, and is configured to further enhance noise reduction performance of the ear tip.

Specifically, the clamping base 130 includes a hollow cylindrical mounting portion 131 protruding from an interior of the first cavity, an outer peripheral side of the hollow cylindrical mounting portion 131 is provided with a clamping protrusion 132, and a peripheral side of the dustproof member or the noise reduction member 140 is provided with a clamping groove 141 adapted to the clamping protrusion 132. During mounting, the clamping groove 141 engages with the clamping protrusion 132, thereby forming a reliable positioning connection. Further, through engagement between the clamping protrusion 132 and the clamping groove 141, the clamping structure may provide moderate assembly resistance and anti-detachment capability, so as to prevent accidental detachment of the dustproof member or the noise reduction member 140 caused by vibration during wearing, movement, or collision, thereby enhancing overall practicality and durability of the structure.

Referring to FIGS. 1 - 5, in this embodiment, one end of the first cavity distal from the through hole 110 is provided with an assembly hole 150 for connection with the noise reduction device 400. The noise reduction device 400 is engaged and inserted into the assembly hole 150 through an inserting post, and positioning and mounting are achieved through engagement and insertion between the inserting post and the assembly hole 150, so that the noise reduction device 400 is stably connected with the ear tip. Further, the support member 200 has a hollow frustoconical shape and is integrally formed with the cap body 100 or the noise reduction device 400. The hollow frustoconical support member 200 has good axial elasticity and volume buffering capability, and can accommodate changes in the ear canal during wearing or compression. Meanwhile, the support member 200 may be integrally formed with the cap body 100 or the noise reduction device 400, so as to make the structural connection more compact, avoid poor sealing or looseness caused by assembly errors, and enhance overall consistency and service life of the product. The structure further includes a wearing portion 500, and the wearing portion 500 is arranged at one end of the noise reduction device 400 distal from the cap body. The wearing portion 500 is a part of the earplug directly contacting the ear auricle of the user, which can adapt to different auricle shapes of users, achieve stable fitting, and prevent loosening or detachment of the earplug. The noise reduction device 400 is configured to process environmental noise.

Embodiment 2: The difference between this embodiment and Embodiment 1 lies in that one end of the cap body 100 distal from the ear canal of the user is provided with a supporting protrusion 120, the inner wall of the supporting protrusion 120 is provided with a plurality of elastic supporting portions (not shown) contacting the support member 200 to provide a supporting function. In this embodiment, a main body of the supporting protrusion does not contact the support member, and the elastic supporting portions provide multi-point flexible support, and enhance overall stability of the device. The elastic structure has a good buffering effect, and can reduce local pressure, improve comfort during long-time wearing, and decrease ear fatigue.

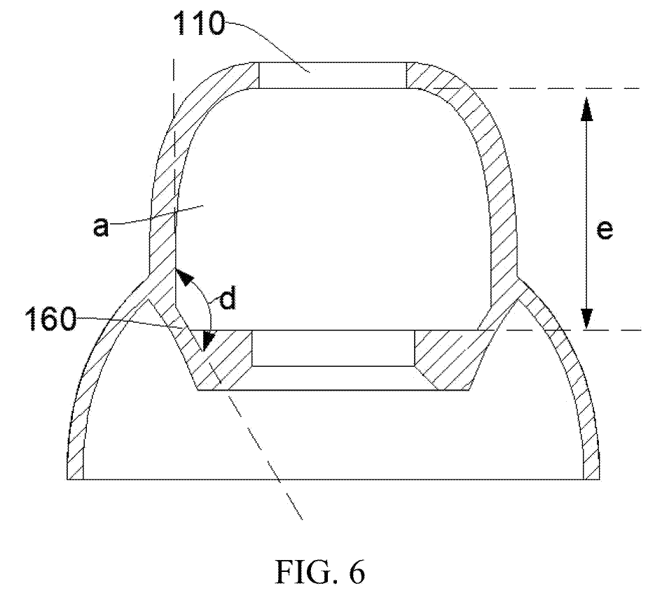

Referring to FIG. 6, Embodiment 3: The difference between this embodiment and Embodiment 1 lies in that this embodiment includes an inclined supporting portion 160. A first end of the inclined supporting portion 160 is connected to a bottom of the first cavity a, and a second end of the inclined supporting portion 160 is connected to a side wall of the first cavity a. A second angle d formed between a line connecting the first end and the second end of the inclined supporting portion 160 and a central axis of the side wall of the first cavity a is 60° to 180°. Further, the inclined supporting portion 160 is arranged between the bottom and the side wall of the first cavity a, so that the ear tip can form a supporting structure under an external force, thereby effectively distributing pressure, preventing excessive collapse of the cavity, and ensuring structural stability of the cap body. The inclined structure provides elastic support and rebound during wearing, enhancing fitting and comfort of the ear tip. The design of the second angle d in a range of 60° to 180° provides high universality and process adaptability. Preferably, the second angle d formed between the line connecting the first end and the second end of the inclined supporting portion 160 and the central axis of the side wall of the first cavity a is 70° to 170°, 80° to 160°, or 90° to 150°, more preferably 60°, 80°, 120°, or 150°. Further, in some embodiments, the central axis of the side wall of the first cavity a may be a straight line connecting a top of the first cavity a and the inclined supporting portion 160.

In this embodiment, a distance e between a top and a bottom of the first cavity a is 3 mm to 8 mm. By defining the distance e as 3 mm to 8 mm, a volume of the acoustic cavity inside the ear tip is maintained within an optimal range balancing acoustic and structural performance. In a case where the distance e is too small, the cavity volume is insufficient, which may result in restricted air flow and suppressed sound wave resonance. In a case where the distance e is too large, the elasticity of the cavity decreases, which may cause high-frequency leakage. By controlling the distance to be within 3 mm to 8 mm, the acoustic cavity is ensured to have an appropriate air buffering space to form good low-frequency resonance, while maintaining the flexibility and resilience of the cavity structure, so that the ear tip is both stable and comfortable during wearing. The distance e may be selected as 4 mm to 7 mm or 5 mm to 6 mm, preferably 4 mm, 6 mm, or 8 mm.

It should be understood that modifications or variations may be made by those skilled in the art based on the above descriptions, and all such modifications and variations are intended to fall within the scope of the appended claims of the present invention.

What is claimed is:

1. An ear tip of an acoustic device, comprising:
a cap body (100), wherein the cap body (100) is made of a flexible material, a first cavity (a) is formed inside the cap body (100), a through hole (110) connected to the first cavity is provided at one end of the cap body (100) close to an ear canal of a user, and an arc-shaped transition portion is formed between one end of the cap body (100) close to the ear canal of the user and a side end connected to the cap body (100).

2. The ear tip of an acoustic device according to claim 1, wherein a distance between a top and a bottom of the first cavity (a) is 3 mm to 8 mm.

3. The ear tip of an acoustic device according to claim 1, comprising an inclined supporting portion (160), wherein a first end of the inclined supporting portion (160) is connected to a bottom of the first cavity (a), a second end of the inclined supporting portion (160) is connected to a side wall of the first cavity (a), and a second angle (d) formed between a line connecting the first end and the second end of the inclined supporting portion (160) and a central axis of the side wall of the first cavity (a) is 60° to 180°.

4. The ear tip of an acoustic device according to claim 1, wherein one end of the cap body (100) distal from the ear canal of the user is provided with a supporting protrusion (120);
the ear tip comprises a support member (200), the support member (200) is in contact with the supporting protrusion (120), and a second cavity (b) is formed between the support member (200) and the supporting protrusion (120), so that air within the second cavity (b) is expelled in a case where the supporting protrusion (120) is pressed by an external force; and
one end of the supporting protrusion (120) distal from the cap body (100) is provided with a leak-preventing contact portion (121) configured to increase a contact area with the support member (200).

5. The ear tip of an acoustic device according to claim 4, wherein a first included angle (c) between the leak-preventing contact portion (121) and the supporting protrusion (120) is 110 degrees to 160 degrees to form an outwardly expanding structure.

6. The ear tip of an acoustic device according to claim 5, wherein the ear tip comprises at least a flexible supporting outer layer and a sound-insulating inner layer which are stacked, and the supporting protrusion (120) is made of the same material as the flexible supporting outer layer and is integrally formed with the flexible supporting outer layer.

7. The ear tip of an acoustic device according to claim 6, wherein a sealing buffer layer is arranged between the supporting protrusion (120) and the flexible supporting outer layer, and the sealing buffer layer is configured to fill gaps, enhance structural integrity, and achieve effective air tightness by fitting a contact interface, so that sound wave leakage is prevented.

8. The ear tip of an acoustic device according to claim 4, wherein one end of the cap body (100) distal from the ear canal of the user is provided with a supporting protrusion (120), and an inner wall of the supporting protrusion (120) is provided with a plurality of elastic supporting portions in contact with the support member (200) to provide a supporting function.

9. The ear tip of an acoustic device according to claim 1, wherein a clamping base (130) is arranged at one end of the through hole (110) close to the first cavity, and a dustproof member or a noise reduction member (140) is clamped onto the clamping base (130).

10. The ear tip of an acoustic device according to claim 9, wherein the clamping base (130) comprises a hollow cylindrical mounting portion (131) protruding from an interior of the first cavity, an outer peripheral side of the hollow cylindrical mounting portion (131) is provided with a clamping protrusion (132), and a peripheral side of the dustproof member or the noise reduction member (140) is provided with a clamping groove (141) adapted to the clamping protrusion (132).

11. A noise reduction earplug, comprising the ear tip of an acoustic device according to claim 1, a noise reduction device (400), and a wearing portion (500), wherein the wearing portion (500) is arranged at one end of the noise reduction device (400) distal from the cap body, and one end of the first cavity distal from the through hole (110) is provided with an assembly hole (150) configured for connection with the noise reduction device (400).

12. The noise reduction earplug according to claim 11, wherein the support member (200) has a hollow frustoconical shape and is integrally formed with the cap body (100) or the noise reduction device (400).
